(19) United States
(12) Patent Application Publication
(10) Pub. No.: US 2016/0081844 A1
(43) Pub. Date: Mar. 24, 2016

(54) Arm sling for an infant

(71) Applicant: Douglas Allen Wagner, Garfield, KS (US)

(72) Inventor: Douglas Allen Wagner, Garfield, KS (US)

(21) Appl. No.: 14/860,456

(22) Filed: Sep. 21, 2015

Related U.S. Application Data
(60) Provisional application No. 62/052,808, filed on Sep. 19, 2014.

(51) Int. Cl. A61F 5/37

(52) U.S. Cl. CPC A61F 5/3738

(57) ABSTRACT

A belt, a band affixed to the front of the belt, and a strap affixed to the side or rear of the belt comprises an arm sling for an infant. The belt wraps around the infant's abdomen and fastens together with a fastening element, such as a hook-and-loop fastener. The infant's arm is inserted into the band in front of the infant. The strap that is attached to the side or rear of the belt then wraps across the upper arm in a loop, and fastens to the belt in front to immobilize the arm against the infant's body.

### CROSS-REFERENCE TO RELATED APPLICATIONS

This Application claims the benefit of U.S. Provisional Application No. 62/052,808, filed Sep. 19, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to medical equipment and supplies, and in particular to an arm sling for an infant. Medical care for infants and toddlers can be especially difficult because they do not understand the need for such things as casts and bandages, and are very uncomfortable, constantly trying to remove them. When a bone is broken, proper support during the healing process is important so that the bone can heal properly. This combination of factors makes the immobilization of a broken arm for an infant or toddler exceptionally challenging. An arm sling for an infant, which comfortably immobilizes the broken arm with a belt around the torso and provides proper support, would resolve this problem.

### SUMMARY OF THE INVENTION

Accordingly, the invention is directed to an arm sling for an infant. The sling is comprised of a belt, a band affixed to the front of the belt, and a strap affixed to the side or rear of the belt. The belt wraps around the infant's abdomen and fastens together with a fastening element, such as a hook-and-loop fastener. The infant's arm is inserted into the band in front of the infant. The strap that is attached to the side or rear of the belt then wraps across the upper arm in a loop, and fastens to the belt in front to immobilize the arm against the infant's body.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

The drawings display the invention configured for wear by an infant with an injury to the left arm. For wear by an infant with an injury to the right arm, the invention may be reconfigured in a mirror image of the displayed configuration.

FIG. 1 is a left side view of the first exemplary embodiment, displaying the belt 10, the band 11, and the strap 12.

FIG. 2 is a front perspective view of the first exemplary embodiment, displaying the belt 10, the band 11, and the strap 12.

FIG. 3 is a right side view of the first exemplary embodiment, displaying the belt 10, the strap 12, and the rivet 13.

FIG. 4 is a rear perspective view of the first exemplary embodiment, displaying the belt 10, the band 11, and the strap 12.

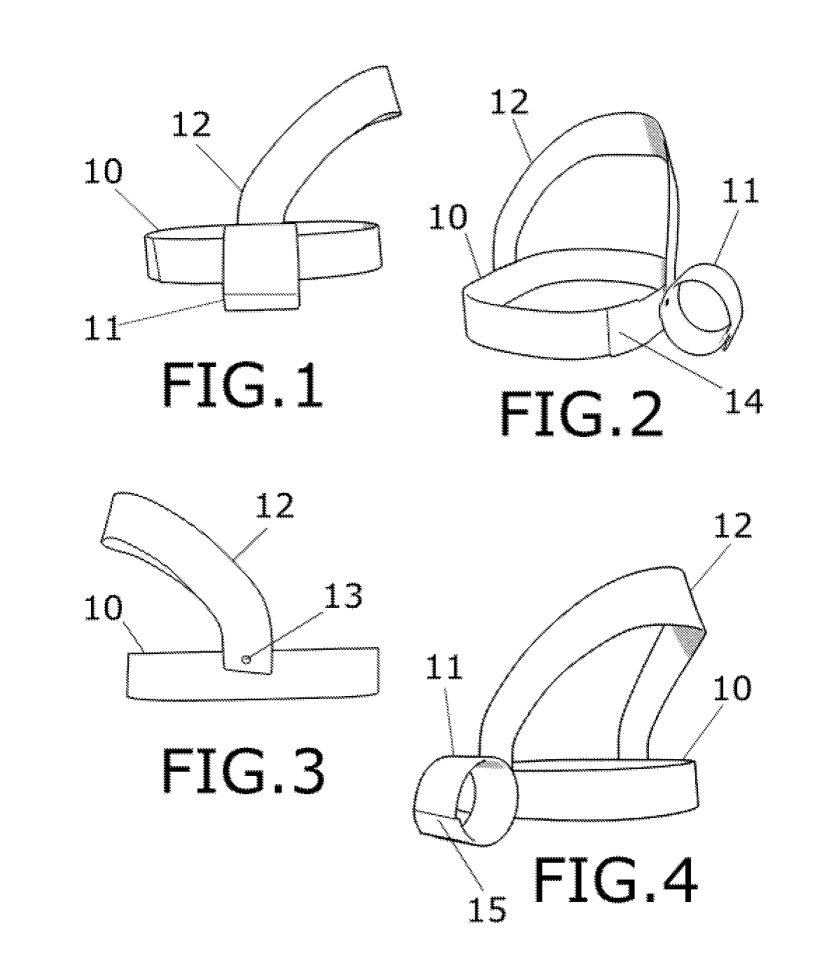

### DETAILED DESCRIPTION OF THE INVENTION

Referring now to the invention in more detail, the invention is directed to an arm sling for an infant.

It is to be understood that while the invention is intended for wear by an infant or toddler with an injured arm, this is not intended as a limitation. With suitable modifications, the invention may be worn by older children or adults, particularly elderly or disabled adults, with arm injuries.

The first exemplary embodiment is comprised of a belt 10, a band 11 affixed to the side of the belt 10, and a strap 12 affixed to the side or rear of the belt 10 with a rivet 13 or similar fastener, such that the strap 12 may be rotated around the attachment point as desired. This enables the adult user to configure the sling for an infant or toddler with an injury to the left arm or the right arm.

To use the first exemplary embodiment, the adult user wraps the belt 10 around the infant's abdomen and fastens it together with a fastening element 14, such as a hook-and-loop fastener, which is provided. The infant's arm is inserted into the band 11 in front of the infant, and the band 11 is also fastened with a fastening element 15, which is provided. The strap 12 that is attached to the side or rear of the belt 10 then wraps across the upper arm in a loop, and fastens to the belt 10 in front to immobilize the arm against the infant's body.

The belt 10, the band 11, and the strap 12 are preferably manufactured from a flexible, durable fabric that is elastic and machine washable, such as elasticized cotton. The rivet 13 is preferably manufactured from a rigid, durable material, such as steel, aluminum alloy, or plastic. The fasteners are preferably manufactured from a semi-rigid, durable material such as plastic or nylon.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. An arm sling for an infant, comprising:
(a) a belt;
(b) a strap;
(c) said strap being affixed to said belt on both ends of said strap;
(d) a band; and
(e) said band forming a loop and being affixed to said belt.

2. The arm sling for an infant of claim 1, wherein one end of said strap is affixed to a front side of said belt and an opposite end of said strap is affixed to a back side of said belt.

3. The arm sling for an infant of claim 1, wherein one end of said strap is affixed to a left side of said belt and an opposite end of said strap is affixed to a right side of said belt.

4. The arm sling for an infant of claim 2, wherein said strap is affixed to said belt via rivet fasteners such that said ends of said strap are configured to rotate around said rivet fastener.

5. The arm sling for an infant of claim 3, wherein said strap is affixed to said belt via rivet fasteners such that said ends of said strap are configured to rotate around said rivet fastener.

6. The arm sling for an infant of claim 1, wherein said belt further comprises a belt fastener affixed to both ends of said belt.

7. The arm sling for an infant of claim 6, wherein said belt fastener is a hook and loop fastener.

8. The arm sling for an infant of claim 1, wherein said band further comprises a band fastener affixed to both ends of said band.

9. The arm sling for an infant of claim 8, wherein said band fastener is a hook and loop fastener.

10. The arm sling for an infant of claim 1, wherein said band is affixed to a front of said belt.

11. The arm sling for an infant of claim 1, wherein said band is affixed to a side of said belt.

12. The arm sling for an infant of claim 8, wherein said band is affixed to a front of said belt.

13. The arm sling for an infant of claim 8, wherein said band is affixed to a side of said belt.

14. The arm sling for an infant of claim 9, wherein said band is affixed to a front of said belt.

15. The arm sling for an infant of claim 9, wherein said band is affixed to a side of said belt.
